(19) United States
(12) Patent Application Publication
(10) Pub. No.: US 2023/0124548 A1
(43) Pub. Date: Apr. 20, 2023

(54) Therapeutic Methods And Uses Thereof

(71) Applicant: Eustralis Pharmaceuticals Limited (Trading As Pressura Neuro), Melbourne, Victoria (AU)

(72) Inventors: Pierre Vankan, Melbourne, Victoria (AU); Peter Pursey, Melbourne, Victoria (AU)

(73) Assignee: Eustralis Pharmaceuticals Limited (Trading As Pressura Neuro), Melbourne, Victoria (AU)

(21) Appl. No.: 17/640,864

(22) PCT Filed: Aug. 21, 2020
(86) PCT No.: PCT/AU2020/050877

§ 371 Date: Mar. 7, 2022

(30) Foreign Application Priority Data
Aug. 23, 2019 (AU) 2019903095

(51) Int. Cl. A61K 31/496; A61K 9/08; A61K 9/00

(52) U.S. Cl. CPC A61K 31/496; A61K 9/08; A61K 9/0019

(57) ABSTRACT

This invention relates generally to therapeutic methods comprising the delivery of particular substituted pyridine based compounds for lowering intracranial pressure (ICP), in treating substance P mediated pathways in the brain such as, but not limited to concussion, post-concussive (or post-concussion) syndrome (PCS), chronic traumatic encephalopathy (CTE), traumatic brain injury (TBI) and stroke.

### CROSS-REFERENCE TO RELATED APPLICATIONS

This is a National Phase entry of International Application No. PCT/AU2020/050877, filed Aug. 21, 2020, which claims priority to Australian Patent Application No. AU20190903095, filed Aug. 23, 2019, the disclosures of which are incorporated herein by reference in their entireties.

### FIELD

This invention relates generally to therapeutic methods comprising the delivery of particular substituted pyridine based compounds for lowering intracranial pressure (ICP), in treating substance P mediated pathways in the brain such as, but not limited to concussion, post-concussive (or post-concussion) syndrome (PCS), chronic traumatic encephalopathy (CTE), traumatic brain injury (TBI) and stroke.

### BACKGROUND

Traumatic brain injury (TBI), also known as intracranial injury, occurs when an external force injures the brain. TBI can be classified based on severity, mechanism (closed or penetrating head injury), or other features (e.g., occurring in a specific location or over a widespread area). TBI can result in physical, cognitive, social, emotional, and behavioural symptoms, and outcome can range from complete recovery to permanent disability or death.

Brain trauma occurs as a consequence of a sudden acceleration or deceleration within the cranium or by a complex combination of both movement and sudden impact. In addition to the damage caused at the moment of injury, a variety of events in the minutes to days following the injury may result in secondary injury. These processes include alterations in cerebral blood flow and the pressure within the skull.

The most common causes of TBI include violence, transportation accidents, construction, and sports. Motor bikes are major causes, increasing in significance in developing countries as other causes reduce. It is estimated that between 1.6 and 3.8 million traumatic brain injuries each year are a result of sports and recreation activities in the US. In children aged two to four, falls are the most common cause of TBI, while in older children traffic accidents compete with falls for this position. TBI is the third most common injury to result from child abuse. Abuse causes 19% of cases of paediatric brain trauma, and the death rate is higher among these cases.

There is a lack of effective medication that can lower elevated intracranial pressure (ICP) in TBI or stroke, neither is there any medication that can prevent the over-expression of hyper-phosphorylated tau protein which has been linked to bad clinical outcome in indications such as TBI but also Alzheimer's disease. Accordingly, there exists a need for a medication that can cure or ameliorate elevated ICP in TBI or stroke or prevent over-expression of hyper-phosphorylated tau protein.

The issue of the lack of effective medication is further compounded by the fact that patients with TBI are likely to be unconscious or may have difficulties swallowing. Accordingly, there is a limitation on how the medication may be administered.

Even while an active pharmaceutical ingredient (API) is identified, there are still many obstacles to overcome in formulating and properly delivering a drug. In formulating a drug suitable for human administration, the skilled person would be aware that the formulation art is not predictable. Various factors need to be carefully investigated and tuned to at least maintain (if not enhance) the pharmacokinetic properties of the API, and/or impart stability to the drug such that it can have an acceptable shelf-life. In this sense, the physical characteristic of the API, the mode of delivery, the flow properties of the composition, the excipient compatibility, the uniformity in production and the release profile needs to be carefully studied and investigated.

The present invention seeks to overcome or ameliorate at least some of the shortcomings of the art in respect to the delivery of specific compounds for lowering ICP.

### SUMMARY OF THE INVENTION

A method of reducing intracranial pressure (ICP) in a subject in need thereof, the method including the step of parenterally (e.g., intravenously) administering an aqueous preparation of a compound of formula (I), or a pharmaceutically acceptable salt thereof,

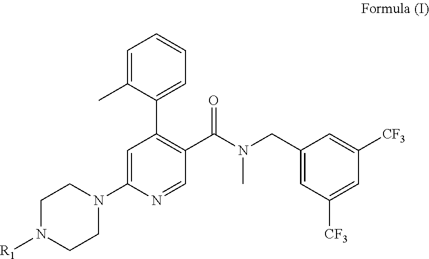

to said subject for up to 30 minutes at a dose level to achieve a Cmax of between 1000 ng/mL.-3000 ng/mL.

Use of a compound of formula (I), or a pharmaceutically acceptable salt thereof,

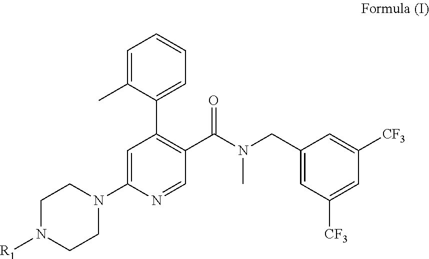

wherein R₁ is H or C1-4 alkyl, in the manufacture of a medicament for reducing intracranial pressure (ICP) in a subject in need thereof, including the step of parenterally administering an aqueous preparation of said medicament to said subject for up to 30 minutes at a dose level to achieve a Cmax of between 1000 ng/mL-3000 ng/mL.

Use of a compound of formula (I), or a pharmaceutically acceptable salt thereof,

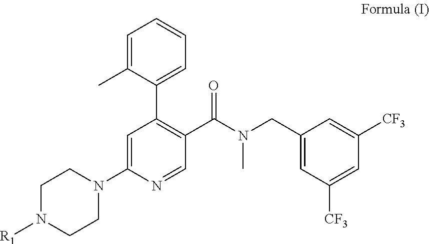

wherein R₁ is H or C1-4 alkyl,

for reducing intracranial pressure (ICP) in a subject in need thereof, including the step of parenterally administering an aqueous preparation of said compound to said subject for up to 30 minutes at a dose level to achieve a Cmax of between 1000 ng/mL-3000 ng/mL.

The present invention provides therapeutic methods that comprises an effective amount of a particular substituted pyridine based compound and other excipients in an aqueous preparation, and more specifically as a parenteral formulation. It advantageously allows administration of the API to a subject in need thereof when the subject is unconscious or unable to swallow, for instance, a subject in need thereof by providing instant relief of substance P mediated processes such as over-expression of hyper-phosphorylated tau protein or elevated intracranial pressure (ICP) and accordingly immediately alleviate the condition and/or symptom of indications as such, but not limited to PCS, CTE, TBI and stroke.

The API mentioned above is a compound of Formula (I), or a pharmaceutically acceptable salt thereof, as represented below

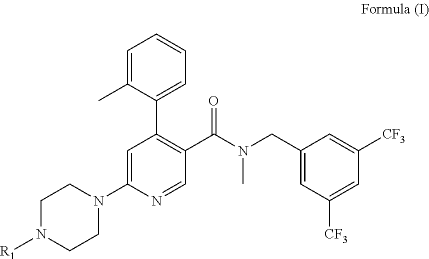

wherein R₁ is H or C₁₋₄ alkyl.

### BRIEF DESCRIPTION OF FIGURES

FIG. 1 is a graph which depicts Intracranial Pressure (ICP) (mmHg) vs time (hrs) in relation to the IV administration of a Compound of Formula (I) 2.HCl (R₁═CH₃).

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Unless defined otherwise, all technical and scientific terms used herein have the same meaning as commonly understood by those of ordinary skill in the art to which the invention belongs. For the purposes of the present invention, the following terms are defined below.

“Alkyl” refers to monovalent alkyl groups which may be straight chained or branched and have from 1 to 4 carbon atoms or more preferably 1 to 3 carbon atoms. As used herein, C₁₋₄ alkyl refers to an alkyl selected from the group consisting of methyl, ethyl, n-propyl, iso-propyl, n-butyl, iso-butyl, sec-butyl and tert-butyl. For instance, in an embodiment R₁ is H or CH₃. In an embodiment R₁ is H. In an embodiment R₁ is CH₃.

“Parenteral” means a mode of administration that occurs elsewhere in the body other than the mouth and the alimentary canal. Accordingly, parenteral administration is administration by delivery via routes other the gastrointestinal tract. As used herein, “parenteral” refers to modes of administration such as intramuscular, intravenous (bolus and/or infusion), subcutaneous, intravesical, or subgingival. In an embodiment, the mode of administration is intravenous.

In an embodiment, R₁ is H, methyl, ethyl, n-propyl, iso-propyl, n-butyl, sec-butyl, iso-butyl or tert-butyl. In another embodiment, R₁ is H, methyl, ethyl, n-propyl or iso-propyl. In another embodiment, R₁ is H. In another embodiment, R₁ is methyl. In another embodiment, R₁ is ethyl. In another embodiment, R₁ is n-propyl.

Accordingly, in some embodiments, the pharmaceutical formulation (ie., aqueous preparation) comprises a compound of Formula (I) or a pharmaceutically acceptable salt, solvate or prodrug thereof selected from the following:

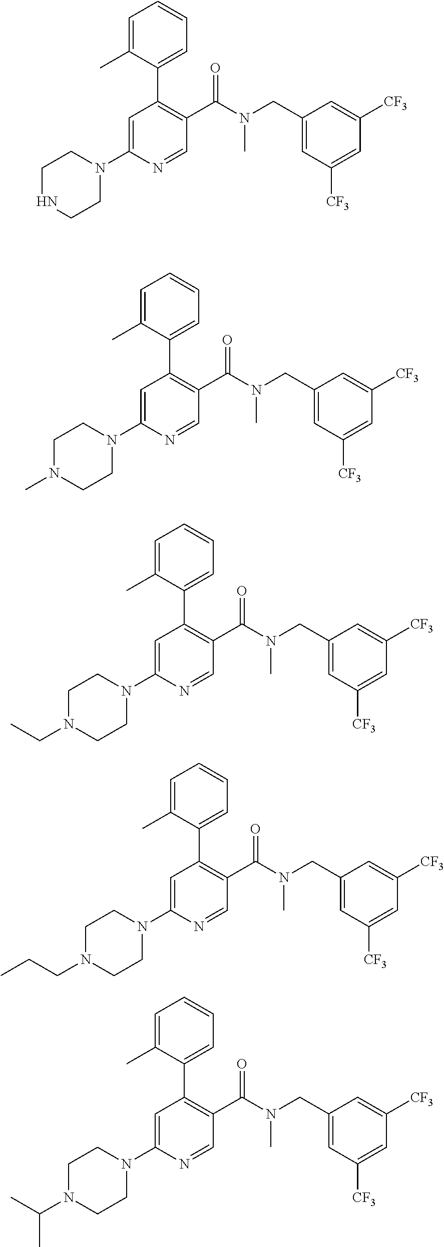 [SMILES: Cc1ccccc1-c1cc(N2CCNCC2)ncc1C(=O)N(C)Cc1cc(C(F)(F)F)cc(C(F)(F)F)c1 ; Cc1ccccc1-c1cc(N2CCN(C)CC2)ncc1C(=O)N(C)Cc1cc(C(F)(F)F)cc(C(F)(F)F)c1 ; CCN1CCN(c2cc(-c3ccccc3C)c(C(=O)N(C)Cc3cc(C(F)(F)F)cc(C(F)(F)F)c3)cn2)CC1 ; CCCN1CCN(c2cc(-c3ccccc3C)c(C(=O)N(C)Cc3cc(C(F)(F)F)cc(C(F)(F)F)c3)cn2)CC1 ; Cc1ccccc1-c1cc(N2CCN(C(C)C)CC2)ncc1C(=O)N(C)Cc1cc(C(F)(F)F)cc(C(F)(F)F)c1]

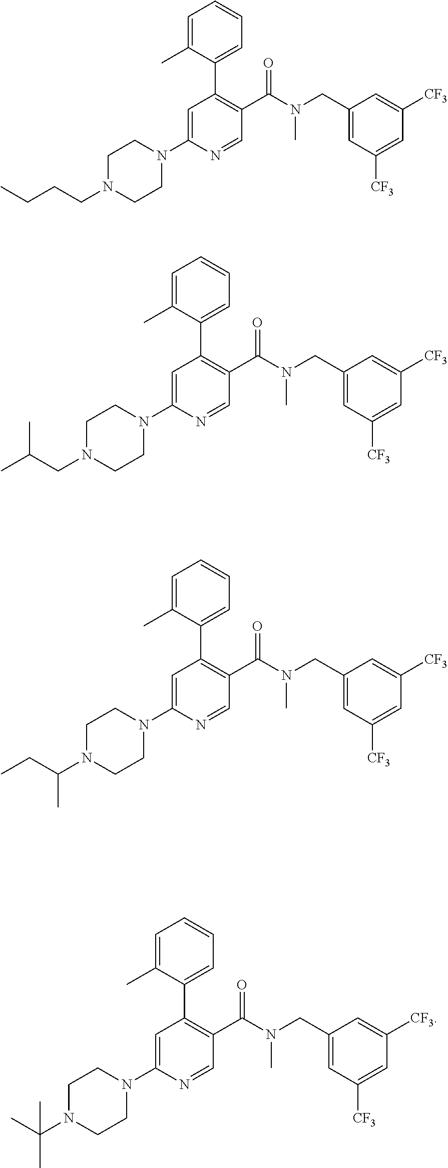 [SMILES: CCCCN1CCN(c2cc(-c3ccccc3C)c(C(=O)N(C)Cc3cc(C(F)(F)F)cc(C(F)(F)F)c3)cn2)CC1 ; Cc1ccccc1-c1cc(N2CCN(CC(C)C)CC2)ncc1C(=O)N(C)Cc1cc(C(F)(F)F)cc(C(F)(F)F)c1 ; CCC(C)N1CCN(c2cc(-c3ccccc3C)c(C(=O)N(C)Cc3cc(C(F)(F)F)cc(C(F)(F)F)c3)cn2)CC1 ; Cc1ccccc1-c1cc(N2CCN(C(C)(C)C)CC2)ncc1C(=O)N(C)Cc1cc(C(F)(F)F)cc(C(F)(F)F)c1]

In particular, in some embodiments, the pharmaceutical formulation comprises a compound of Formula (I) or a pharmaceutically acceptable salt, solvate or prodrug thereof which is:

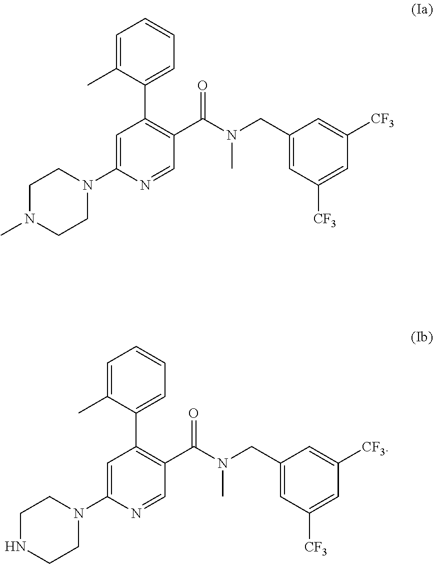 [SMILES: Cc1ccccc1-c1cc(N2CCN(C)CC2)ncc1C(=O)N(C)Cc1cc(C(F)(F)F)cc(C(F)(F)F)c1 ; Cc1ccccc1-c1cc(N2CCNCC2)ncc1C(=O)N(C)Cc1cc(C(F)(F)F)cc(C(F)(F)F)c1]

In an embodiment, compound of Formula (I) or a pharmaceutically acceptable salt, solvate or prodrug thereof is provided as a salt. In another embodiment, compound of Formula (I) or a pharmaceutically acceptable salt, solvate or prodrug thereof is a HCl salt. In another embodiment, compound of Formula (I) or a pharmaceutically acceptable salt, solvate or prodrug thereof is a 2HCl salt. Accordingly, in some embodiments, the pharmaceutical composition comprises a compound of Formula (I) or a pharmaceutically acceptable salt, solvate or prodrug thereof selected from the following:

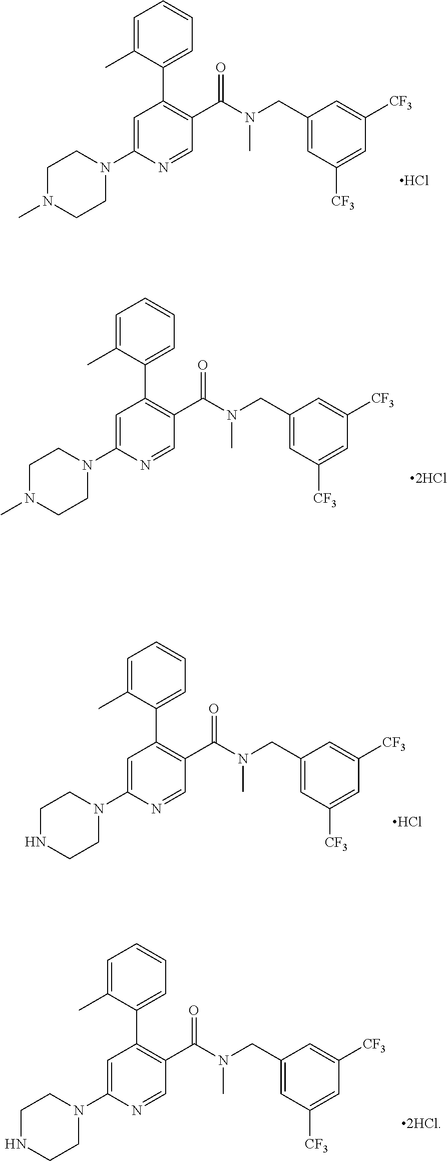

In the development of the compound of Formula (I) experiments consistently showed strong efficacy of the compound to blocking central NK1 receptors.

In order to avoid any potential safety issues, the infusion in human clinic trials was slowed down to 2 hours to generate the same AUC but a lower Cmax.

This was based on the opinion that for the blockade of the receptor, a minimum plasma concentration is needed to be maintained to allow sufficient concentration of the compound for a functional receptor blocking activity.

However two patients were treated in a clinical trial, using a slow infusion rate over a timespan of 2 hours. The ICP curves in these two patients continued to show marked fluctuations including periods of significant elevation in ICP suggesting no or minimal efficacy—when given as a 2-hour infusion.

A pharmacodynamic study to assess functional blocking of central NK1 receptors was performed in healthy volunteers. This was done by testing the efficacy of the compound in blocking apomorphine induced emesis. This study indicates the need for a minimum concentration of the initial peak at infusion to obtain efficacious levels, rather than minimal plasma-levels at the time of apomorphine challenge. These levels are in the range of 1000-1300 ng/ml of Cmax, whereas the plasma-levels obtained with the 2 hr infusion schedule were in the range between 400 and 900 ng/ml.

In the phase 1 apomorphine challenge study the ability of the compound to block emesis induced by apomorphine was tested in healthy volunteers. It was found that when subjects (n=5) were first treated with 90 mg of the compound and then challenged after 2 hours with apomorphine, an average of 1.4 “retches and vomits” was observed, which indicated full efficacy for NK1 receptor antagonism (efficacy was defined as “reducing the average number of retches and vomits below 3, without need for rescue, anti-emetic, therapy). Similarly, when the apomorphine challenge was performed 20 hrs after 90 mg of Compound of Formula (I) R₁=CH₃dosing (n=5), the efficacy was only slightly reduced, with an average of 2.6 retches and vomits observed. At neither time point did subjects require rescue medication to prevent uncontrolled vomiting. On the other hand, when the apomorphine challenge was performed 2 hours after a 30 mg dose (n=5), an average of 4.2 retches and vomits were observed, which would have been even higher had two of five subjects not received rescue medication to block the apomorphine effects. Importantly the plasma concentration of the Compound 2 hours after the 30 mg dose was 60% higher than the plasma concentration 20 hours after the 90 mg dose. Nevertheless it showed a lower efficacy.

TABLE 1
Blocking of apomorphine induced emesis with Compound of Formula (I) 2HCl (R₁ = CH₃)

| Compound | Cmax | | | | Average |
|---|---|---|---|---|---|
| Dose/Time to | at end of | Conc. at | Number of | Number of | Number of |
| apomorphine | infusion | challenge | Retches | Vomits | Retches or |
| challenge | (ng/ml) | (ng/ml) | (Subjects/Events) | (Subjects/Events) | Vomits |
| 30 mg/2 h | 252 | 112 | 2/9 | 3/>12* | 4.2 |
| (N = 5) | | | | | |
| 90 mg/2 h | 1045 | 356 | 2/5 | 2/2 | 1.4 |
| (N = 5) | | | | | |
| 90 mg/20 h | 1273 | 69 | 4/12 | 1/1 | 2.6 |
| (N = 5) | | | | | |

*2 of 5 subjects required rescue therapy for uncontrolled vomiting, as per protocol.

These data indicate that a minimum average peak level in the range of at least 1000 ng/ml to 1300 ng/ml are preferred for central NK1 blockade and that, once blockade is achieved the reduction of adverse effect (retches/vomits) lasts up to a minimum of 20 hrs. These levels are obtained using a shorter 15-30 minute infusion.

The inventors surmise that this can only be explained by assuming that the high initial peak is crucial for blocking the NK1 receptor with the compound, rather than the maintenance of trough levels.

On the other hand it is postulated that for safety reasons Cmax should not exceed levels above 3000 ng/ml, in order to maintain a safety margin of at least 10 fold over the No-Observed-Adverse-Effect Level (NOAEL) observed in toxicology studies in other animal studies.

The present inventors believe that the need for a high peak concentration to obtain efficacy can be explained by assuming that a minimum peak at Cmax is required to allow passage of a sufficient amount of the compound into the brain. Additionally, because the compound is a non-competitive inhibitor of the NK1 receptor the compound remains active for a longer period of time also when plasma and brain concentrations drop to lower levels. In this model a high initial peak is needed to rapidly enter the brain allowing it to block the receptor both initially and over a longer time after dosing.

An alternative way of administration would be a bolus injection rather than a 15 or 30 minute infusion, with a lower dose, that achieves an equally high initial Cmax, but which will have a lower AUC.

For efficacy the compound of Formula (I) is preferably administered in a short infusion to allow a minimal peak level or Cmax between 1000 ng/ml and 3000 ng/ml.

This can be achieved by a short infusion for up to 30 minutes with doses up to 200 mg of the compound of Formula (I) dihydrochloride salt equivalent to about 190 mg of free base when administered via IV.

In another embodiment this can be achieved by a short infusion for up to 30 minutes with doses up to about 150 mg of the compound of Formula (I) dihydrochloride salt equivalent to about 140 mg of free base when administered via IV.

In another embodiment this can be achieved by a short infusion for up to 30 minutes with doses up to about 90 mg of the compound of Formula (I) dihydrochloride salt equivalent to about 80 mg of free base when administered via IV.

This can be achieved by a short infusion of for up to 30 minutes with doses up to about 90 mg of the compound of Formula (I) dihydrochloride salt equivalent to about 80 mg of free base when administered via IV.

Alternatively this can be achieved by an injection, instead of an infusion, of a lower dose, targeting the same Cmax between 1000 and 3000 ng/ml. Such a lower dose can be in the range of about 1-40 mg/kg.

This can be achieved by injection doses of 1 mg to about 40 mg of the compound of Formula (I) dihydrochloride salt (equivalent to about 30 mg/kg of free base). The higher rate of infusion will generate a higher or equally high Cmax while using a lower dose.

In an embodiment, the parenteral, pharmaceutical composition is an intravenous, pharmaceutical composition. In another embodiment, the composition is an intravenous bolus, pharmaceutical composition. In another embodiment, the composition is an intravenous infusion, pharmaceutical composition. In another embodiment, the composition is an intramuscular, pharmaceutical composition. In another embodiment, the composition is a subcutaneous, pharmaceutical composition. In another embodiment, the composition is an intravesical, pharmaceutical composition. In another embodiment, the composition is a subgingival, pharmaceutical composition.

In an embodiment, the reconstitutable, parenteral, pharmaceutical composition or parenteral, pharmaceutical composition is subjected to sterilisation. In another embodiment, the composition is subjected to gamma radiation. In another embodiment, the composition is subjected to heat treatment. In another embodiment, the composition is subjected to moist heat treatment. For example, the composition may be heat treated at about 140° C., about 130° C., about 120° C., about 110° C., about 100° C., or about 90° C. The composition may be heat treated for about 5 min, about 10 min, about 15 min, about 20 min, about 30 min, about 40 min, about 50 min, about 60 min or about 120 min.

In one embodiment, the dosage of the pharmaceutical composition administered to a subject in the various embodiments of the present invention is such that compound of Formula (I) is administered in the range from about 1 mg to 200 mg depending on administration route as discussed above. In one embodiment, the dosage of the pharmaceutical formulation administered to a subject in the various embodiments of the present invention is such that compound of Formula (I), or pharmaceutically acceptable salt thereof, is administered in the range from about 1.0 mg, 2.0 mg, 3.0 mg, 4.0 mg, 5.0 mg, 6.0 mg, 7.0 mg, 8.0 mg, 9.0 mg, 10.0 mg, 11.0 mg, 12.0 mg, 13.0 mg, 14.0 mg, 15.0 mg, 16.0 10 mg, 17.0 mg, 18.0 mg, 19.0 mg, 20.0 mg, 21.0 mg, 22.0 mg, 23.0 mg, 24.0 mg, 25.0 mg, 26.0 mg, 27.0 mg, 28.0 mg, 29.0 mg, 30.0 mg, 31.0 mg, 32.0 mg, 33.0 mg, 34.0 mg, 35.0 mg, 36.0 mg, 37.0 mg, 38.0 mg, 39.0 mg, 40.0 mg, 41.0 mg, 42.0 mg, 43.0 mg, 44.0 mg, 45.0 mg, 46.0 mg, 47.0 mg, 48.0 mg, 49.0 mg, 50.0 mg, 51.0 mg, 52.0 mg, 53.0 mg, 54.0 mg, 55.0 mg, 56.0 mg, 57.0 mg, 58.0 mg, 59.0 mg, 60.0 mg, 61.0 mg, 62.0 mg, 63.0 mg, 64.0 mg, 65.0 mg, 66.0 mg, 67.0 mg, 68.0 mg, 69.0 mg, 70.0 mg, 71.0 mg, 72.0 mg, 73.0 mg, 74.0 mg, 75.0 mg, 76.0 mg, 77.0 mg, 78.0 mg, 79.0 mg, 80.0 mg, 81.0 mg, 82.0 mg, 83.0 mg, 84.0 mg, 85.0 mg, 86.0 mg, 87.0 mg, 88.0 mg, 89.0 mg, 90.0 mg, 91.0 mg, 92.0 mg, 93.0 mg, 94.0 mg, 95.0 mg, 96.0 mg, 97.0 mg, 98.0 mg, 99.0 mg, 100 mg, 105 mg, 110 mg, 120 mg, 130 mg, 140 mg, 150 mg, 160 mg, 170 mg, 180 mg, 190 mg, or 200 mg, or any range within the above amounts.

In the embodiment wherein the administration route is intravenous the present invention is such that compound of Formula (I), or pharmaceutically acceptable salt thereof, is administered in the range from about 60.0 mg, 61.0 mg, 62.0 mg, 63.0 mg, 64.0 mg, 65.0 mg, 66.0 mg, 67.0 mg, 68.0 mg, 69.0 mg, 70.0 mg, 71.0 mg, 72.0 mg, 73.0 mg, 74.0 mg, 75.0 mg, 76.0 mg, 77.0 mg, 78.0 mg, 79.0 mg, 80.0 mg, 81.0 mg, 82.0 mg, 83.0 mg, 84.0 mg, 85.0 mg, 86.0 mg, 87.0 mg, 88.0 mg, 89.0 mg, 90.0 mg, 91.0 mg, 92.0 mg, 93.0 mg, 94.0 mg, 95.0 mg, 96.0 mg, 97.0 mg, 98.0 mg, 99.0 mg, 100 mg, 105 mg, 110 mg, 120 mg, 130 mg, 140 mg, 150 mg, 160 mg, 170 mg, 180 mg, 190 mg, or 200 mg, or any range within the above amounts.

In the embodiment wherein the administration route is bolus injection the present invention is such that compound of Formula (I), or pharmaceutically acceptable salt thereof, is administered in the range from about 1.0 mg, 2.0 mg, 3.0 mg, 4.0 mg, 5.0 mg, 6.0 mg, 7.0 mg, 8.0 mg, 9.0 mg, 10.0 mg, 11.0 mg, 12.0 mg, 13.0 mg, 14.0 mg, 15.0 mg, 16.0 10 mg, 17.0 mg, 18.0 mg, 19.0 mg, 20.0 mg, 21.0 mg, 22.0 mg, 23.0 mg, 24.0 mg, 25.0 mg, 26.0 mg, 27.0 mg, 28.0 mg, 29.0 mg, 30.0 mg, 31.0 mg, 32.0 mg, 33.0 mg, 34.0 mg, 35.0 mg, 36.0 mg, 37.0 mg, 38.0 mg, 39.0 mg, to 40.0 mg, or any range within the above amounts.

In an embodiment the effective amount is administered as a single or multiple dose. In an embodiment the effective amount is administered as a single or multiple intravenous dose. In an embodiment the effective amount is administered as a single or multiple injection dose.

In certain embodiments the administration method as disclosed herein is repeated at least 1 more time about from 2-6 hours from first administration.

In certain embodiments the administration method is repeated at least 1 more time about 4 hours from first administration.

In certain embodiments the administration method is repeated at least 2 more time about 2-8 hours from first administration.

In certain embodiments the administration method is repeated at least 2 more times about 4 and 8 hours from first administration

In certain embodiments the administration method is repeated at least 3 more times about from 3-18 hours from first administration.

In certain embodiments the administration method is repeated at least 3 more times about 4, 8 and 12 hours from first administration.

In certain embodiments the administration method is repeated at least 1 more time about 4 hours from the first administration for two to four consecutive days.

In certain embodiments the subject receives the administration method within 1-48 hrs after being involved in a TBI or having a stroke, or any other incident or disease condition which elevates the subjects ICP levels above 20 mmHg Without wishing to be bound by any particular theory the present inventors believe that the ability of the present compound to block the increase in substance P (and therefore reduce ICP in an effective manner) may be compromised if the patient doesn't receive the first administration dose within the first 48 hrs after being involved in a TBI or having a stroke, or any other incident or disease condition which elevates the subjects ICP levels above 20 mmHg

The skilled person would appreciate that the aim of the present administration regime is to provide effective stabilisation of ICP of the subject in need thereof. In this regard, effective stabilisation is deemed to have been achieved once an ICP level of below 20 mmHg is established from 5 to over 10 hrs after the last administration dose is provided to said subject (patient), for instance, over 5 hrs, over 6 hrs, over 7 hrs, over 8 hrs, over 9 hrs, or over 10 hrs.

In an embodiment, the method for treating elevated intracranial pressure is also a method for treating traumatic brain injury as ICP is a symptom associated with TBI

In another embodiment, the method for treating elevated intracranial pressure is also a method for treating stroke as ICP is a symptom associated with stroke

In an embodiment the pharmaceutical formulation is to be administered as a treatment for injury associated with concussion post the injury event which is associated with an increase in ICP.

Thus the terms “treat,” “treatment,” and “treating” also refers to one or more of the following:
(a) relieving or alleviating at least one symptom of a disorder in a subject, including reducing intracranial pressure in a TBI patient;
(b) relieving or alleviating the intensity and/or duration of a manifestation of a disorder experienced by a subject including, but not limited to, those that are in response to a given stimulus (e.g., pressure, tissue injury, cold temperature, etc.); and
(c) arresting, delaying the onset (i.e., the period prior to clinical manifestation of a disorder) and/or reducing the risk of developing or worsening a disorder.

A subject or patient in whom administration of the therapeutic compound is an effective therapeutic regimen for a disease or disorder is preferably a human

In certain embodiments the human subject (patient) is selected with a presented ICP of above 25 mmHg, such as above 26 mmHg, above 27 mmHg, above 28 mmHg, above 29 mmHg, above 30 mmHg, above 31 mmHg, above 32 mmHg, above 33 mmHg, above 34 mmHg, above 35 mmHg, above 36 mmHg, above 37 mmHg, above 38 mmHg, above 39 mmHg, above 40 mmHg, above 41 mmHg, above 42 mmHg, above 43 mmHg, or above 44 mmHg

In certain embodiments the subject (patient) presents with a Glascow Coma Scale of 3-12.

Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

The parental formulations may contain any other suitable carriers, diluents or excipients. These include all conventional solvents, dispersion media, fillers, solid carriers, coatings, antifungal and antibacterial agents, surfactants, isotonic and absorption agents and the like. It will be understood that the compositions of the invention may also include other supplementary physiologically active agents.

For example, the pharmaceutical formulation may further comprise a preservative, a buffer, stabiliser and/or a viscosity enhancing agent. Examples of suitable preservatives are benzoic acid esters of para-hydroxybenzoic acid, phenols, phenylethyl alchohol or benzyl alcohol.

Examples of suitable buffers are sodium phosphate salts, citric acid, tartaric acid and the like. Examples of suitable stabilisers are, antioxidants such as alpha-tocopherol acetate, alpha-thioglycerin, sodium metabisulphite, ascorbic acid, acetylcysteine, 8-hydroxyquinoline, chelating agents such as disodium edentate. Examples of suitable viscosity enhancing agents, suspending or dispersing agents are substituted cellulose ethers, substituted cellulose esters, polyvinyl alchohol, polyvinylpyrrolidone, carbomer, polyoxypropylene glycols, and sorbitan sesquioleate.

For example, the pharmaceutical formulation may further comprise a pH controller. Examples of suitable pH controllers include hydrochloric acid, sodium hydroxide and the like.

It will be appreciated that any compound that is a prodrug of a compound of formula (I) is also within the scope and spirit of the invention. The term “pro-drug” is used in its broadest sense and encompasses those derivatives that are converted in vivo to the compounds of the invention. Such derivatives would readily occur to those skilled in the art, and include, for example, phosphonic acid derivatives.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### Examples

Compound of Formula (I), in particular compound (Ia) as shown below, is used in all examples, and in particular the 2HCl salt of compound (Ia) (Compound (Ia) HCl).

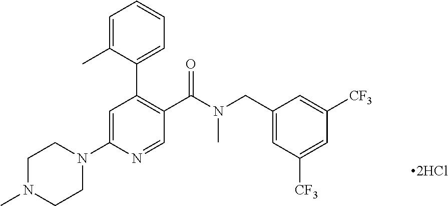 [SMILES: Cc1ccccc1-c1cc(N2CCN(C)CC2)ncc1C(=O)N(C)Cc1cc(C(F)(F)F)cc(C(F)(F)F)c1 ; "·2HCl"]

(Designated herein as “EUC-001”)

Solubility

Table 3 shows a solubility assessment of compound (Ia) and its respective pH. Ethanol was also assessed as part of this study. The API was freely soluble at a concentration of 50 mg/mL of Compound (Ia) HCl, where a golden yellow colour was observed.

TABLE 3
Solubility of Compound (Ia) 2HCl in Water

| Weight of | | | | | |
|---|---|---|---|---|---|
| Compound | Volume of | | | | |
| (Ia) HCl | Water | Concentration | | Appearance | |
| (mg) | (mL) | (mg/mL) | Result | of solution | pH |
| 50 | 10 | 5 | Very soluble | Light yellow | 2.39 |
| 100 | 10 | 10 | Very soluble | Light yellow | 2.14 |
| 150 | 10 | 15 | Soluble | Light yellow | 2.02 |
| 200 | 10 | 20 | Soluble | Yellow | 1.87 |
| 250 | 10 | 25 | Soluble | Yellow | 1.81 |
| 300 | 10 | 30 | Soluble | Yellow | 1.75 |
| 350 | 10 | 35 | Soluble | Yellow | 1.72 |
| 400 | 10 | 40 | Soluble | Gold yellow | 1.66 |
| 450 | 10 | 45 | Soluble | Gold yellow | 1.54 |
| 500 | 10 | 50 | Soluble | Gold yellow | 1.52 |

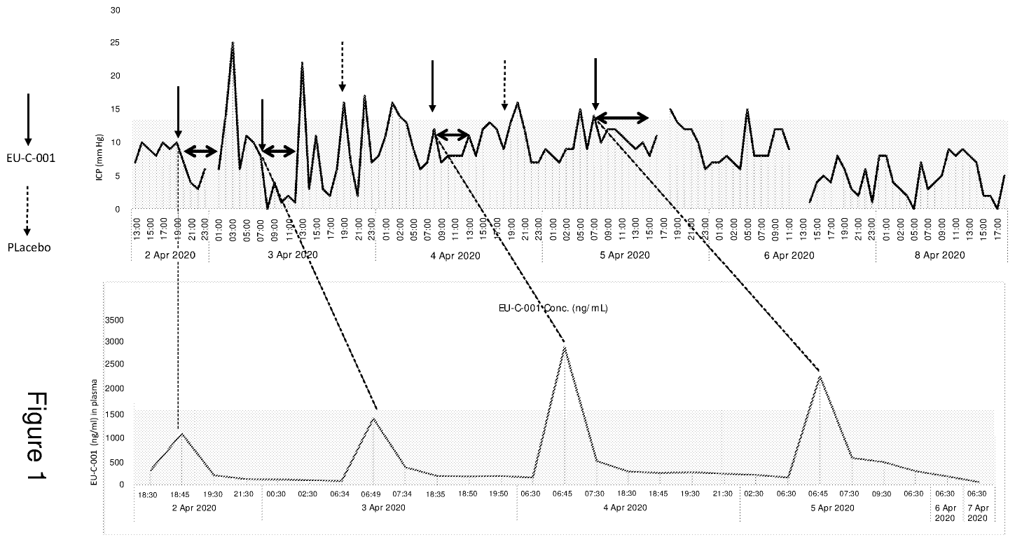

EU-C-001 was formulated as a stock solution consisting of 15 mg/ml EU-C-001 in 80% propylene glycol and 20% water for injection set to pH 4.5-5.5 with sodium hydroxide. Placebo stock solution was identical without the active substance. The stock solution was filled into vials containing 7.5 ml. For administration 6 ml of stock solution (90 mg) was infused in a commercially available 5% glucose solution. The solution was infused over 15 minutes by IV. Administration of EU-C-001 was done at t=0 hrs, t=12 hrs, t=36 hrs and t=60 hrs. Placebo was administered at t=24 hrs and t=48 hrs. Patients in the study had an intracranial pressure monitor inserted in the skull as part of standard treatment. A rise of ICP above 20 mm Hg as measured with the intracranial monitor was an inclusion criterion for the study. ICP was recorded hourly for 5 days following the start of treatment. The results are provided in FIG. 1.

1. A method of reducing intracranial pressure (ICP) in a subject in need thereof, the method including the step of parenterally administering an aqueous preparation of a compound of formula (I), or a pharmaceutically acceptable salt thereof, 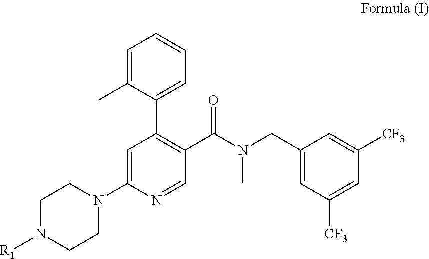
wherein R₁ is H or C₁₋₄ alkyl,
to said subject for up to 30 minutes at a dose level to achieve a Cmax of between 1000 ng/mL-3000 ng/mL.

2. The method according to claim 1, wherein the parenteral administration step is intravenous administration.

3. The method according to claim 2, wherein administration is for up to about 15 minutes.

4. The method according to claim 3, wherein the Cmax of between 1000 ng/mL-3000 ng/mL is achieved at a dose of a compound of Formula (I), or a pharmaceutically acceptable salt thereof, from about 30 mg to 200 mg.

5. The method according to claim 4, wherein the Cmax of between 1000 ng/mL-3000 ng/mL is achieved at a dose of a compound of Formula (I), or a pharmaceutically acceptable salt thereof, from about 90 mg to 150 mg.

6. The method according to claim 1, wherein the method is repeated at least 1 more time about 4 hours from the first administration.

7. The method according to claim 1, wherein the method is repeated at least 1 more time about 4 hours from the first administration for two to four consecutive days.

8. The method according to claim 1, wherein the parenteral administration step is by injection.

9. The method according to claim 8, wherein the Cmax of between 1000 ng/mL-3000 ng/mL is achieved at a dose of a compound of Formula (I), or a pharmaceutically acceptable salt thereof, from about 1 mg to 40 mg.

10. The method according to claim 8, wherein the method is repeated at least 1 more time about 4 hours from the first injection.

11. The method according to claim 1, wherein the compound of Formula (I) is selected from:

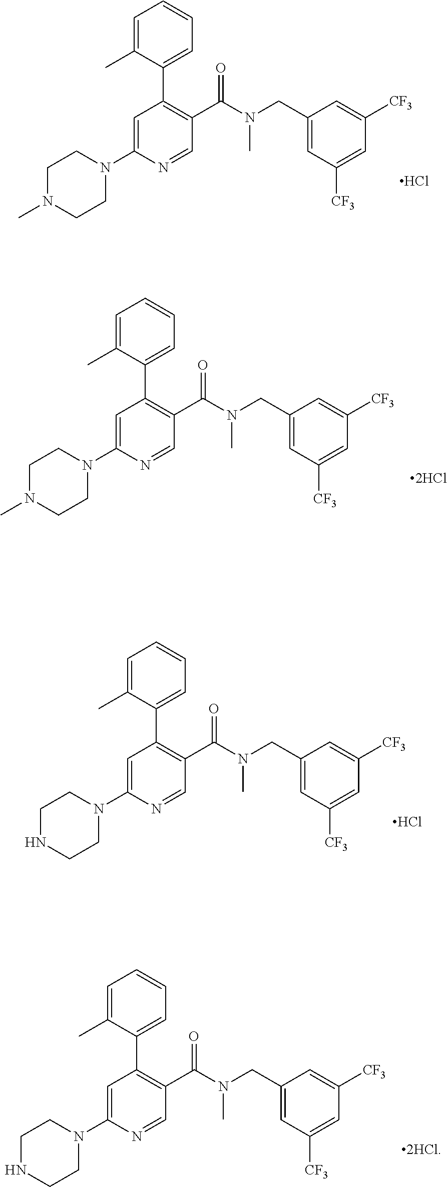

12. The method according to claim 11, wherein the compound of Formula (I) is

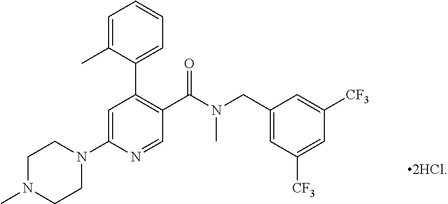 [SMILES: Cc1ccccc1-c1cc(N2CCN(C)CC2)ncc1C(=O)N(C)Cc1cc(C(F)(F)F)cc(C(F)(F)F)c1 ; "·2HCl."]

13. The method according to claim 1, wherein the method achieves a reduction of ICP of below 20 mmHg.

14. The method according to claim 13, wherein the method achieves a reduction of ICP of below 20 mmHg for 5-10 hrs.

15. The method according to claim 1, wherein the subject presents with an ICP level of above 20 mm Hg.

16. The method according to claim 1, wherein the subject receives the administration method within 1-48 hrs after being involved in a TBI or having a stroke.

17. A method of manufacturing a medicament for reducing intracranial pressure (ICP) in a subject in need thereof, including the step of parenterally administering an aqueous preparation of the medicament comprising a compound of formula (I), or a pharmaceutically acceptable salt thereof, 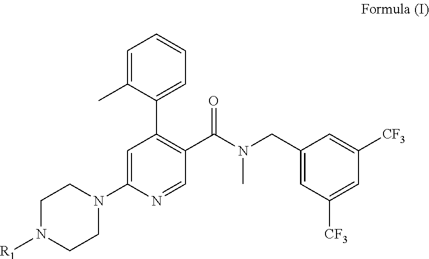
wherein R₁ is H or C₁₋₄ alkyl,
to said subject for up to 30 minutes at a dose level to achieve a Cmax of between 1000 ng/mL-3000 ng/mL.

18. A method of reducing intracranial pressure (ICP) in a subject in need thereof, including the step of parenterally administering an aqueous preparation of a compound of formula (I), or a pharmaceutically acceptable salt thereof, 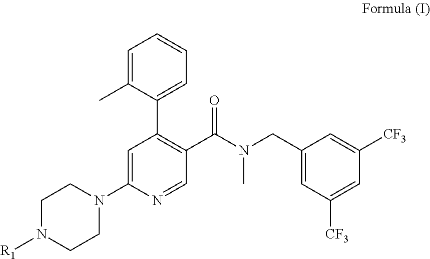
wherein R₁ is H or C₁₋₄ alkyl,
to said subject for up to 30 minutes at a dose level to achieve a Cmax of between 1000 ng/mL-3000 ng/mL.
